(19) United States
(12) Patent Application Publication
(10) Pub. No.: US 2015/0010681 A1
(43) Pub. Date: Jan. 8, 2015

(54) CULTIVATED ORYZA SATIVA PLANT HAVING A PARTIALLY OR FULLY MULTIPLIED GENOME AND USES OF SAME

(71) Applicant: Kailma Bio Agritech Ltd., Kfar-Tavor (IL)

(72) Inventors: Amit Avidov, Kiryan-Tivon (IL); Moran Shelef, Moshav Shadmot Dvora (IL); Limor Baruch, Moshav Tel Adashim (IL); Lilah Rothem, Kibbutz Megiddo (IL)

(21) Appl. No.: 14/380,150

(22) PCT Filed: Feb. 19, 2013
(86) PCT No.: PCT/IL2013/050145

§ 371 Date: Aug. 21, 2014

Related U.S. Application Data
(60) Provisional application No. 61/601,645, filed on Feb. 22, 2012.

(51) Int. Cl. A01H 1/08; C12N 15/01; A23L 1/10; A01H 5/10

(52) U.S. Cl. CPC A01H 1/08; A01H 5/10; C12N 15/01; A23L 1/1041; A23V 2002/00
USPC 426/443; 800/320.2; 800/260; 800/276

(57) ABSTRACT

A cultivated Oryza sativa plant is provided. The cultivated Oryza sativa plant has a partially or fully multiplied genome being at least as fertile as a diploid Oryza sativa plant isogenic to said genomically multiplied Oryza sativa plant when grown under the same conditions.

FIELD AND BACKGROUND OF THE INVENTION

The present invention, in some embodiments thereof, relates to a cultivated Oryza sativa plant having a partially or fully multiplied genome and uses of same.

Rice is the seed of the monocot plants Oryza sativa (Asian rice) or Oryza glaberrima (African rice). Cultivated rice is diploid (2N=24). There are some wild species which are tetraploid as described by Li et al. 2000 International Rice Research Notes 25:19-22. As a cereal grain, it is the most important staple food for a large part of the world's human population, especially in East Asia, Southeast Asia, South Asia, the Middle East, and the West Indies. It is the grain with the third-highest worldwide production, after maize (corn) and wheat, according to data for 2009.

Since a large portion of maize crops are grown for purposes other than human consumption, rice is the most important grain with regard to human nutrition and caloric intake, providing more than one fifth of the calories consumed worldwide by the human species.

Rice is normally grown as an annual plant, although in tropical areas it can survive as a perennial and can produce a ratoon crop for up to 30 years. The rice plant can grow to 1-1.8 m (3.3-5.9 ft) tall, occasionally more depending on the variety and soil fertility. It has long, slender leaves 50-100 cm (20-39 in) long and 2-2.5 cm (0.79-0.98 in) broad. The small wind-pollinated flowers are produced in a branched arching to pendulous inflorescence 30-50 cm (12-20 in) long. The edible seed is a grain (caryopsis) 5-12 mm (0.20-0.47 in) long and 2-3 mm (0.079-0.12 in) thick.

Rice cultivation is well-suited to countries and regions with low labor costs and high rainfall, as it is labor-intensive to cultivate and requires ample water. Rice can be grown practically anywhere, even on a steep hill or mountain. Although its parent species are native to South Asia and certain parts of Africa, centuries of trade and exportation have made it commonplace in many cultures worldwide.

Today, the majority of all rice produced comes from China, India, Indonesia, Pakistan, Bangladesh, Vietnam, Thailand, Myanmar, Philippines, and Japan. Asian farmers still account for 92% of the world's total rice production.

World production of rice has risen steadily from about 200 million tonnes of paddy rice in 1960 to over 678 million tonnes in 2009. However, rice production is still lagging. This gap stems from inferior farming system technology and knowledge, in countries like India. In addition many rice grain producing countries have significant losses post-harvest at the farm and because of poor roads, inadequate storage technologies, inefficient supply chains and farmer's inability to bring the produce into retail markets dominated by small shopkeepers. A World Bank—FAO study claims 8% to 26% of rice is lost in developing nations, on average, every year, because of post-harvest problems and poor infrastructure. Some sources claim the post-harvest losses to exceed 40%. Not only do these losses reduce food security in the world, the study claims that farmers in developing countries such as China, India and others loose approximately US$89 billion of income in preventable post-harvest farm losses, poor transport, the lack of proper storage and retail. One study claims that if these post-harvest grain losses could be eliminated with better infrastructure and retail network, in India alone enough food would be saved every year to feed 70 to 100 million people over a year.

A recent study found that, as a result of rising temperatures and decreasing solar radiation during the later years of the 20th century, the rice yield growth rate has decreased in many parts of Asia, compared to what would have been observed had the temperature and solar radiation trends not occurred. The yield growth rate had fallen 10-20% at some locations. The study was based on records from 227 farms in Thailand, Vietnam, Nepal, India, China, Bangladesh, and Pakistan. The mechanism of this falling yield is not clear but might involve increased respiration during warm nights, so expending energy without being able to photosynthesize.

Thus, a continuing goal of plant breeders is to develop stable high yielding rice varieties that are agronomically advantageous.

Until recently, genetic improvement of rice for agronomic and quality traits has been carried out by traditional plant breeding methods and improved cultural management practices. Advances in tissue culture and transformation technologies have resulted in the production of transgenic plants of all major cereals, including rice. Induction of polyploidy has been attempted as means for improving plant yield and quality.

He et al. Planta 2010 232:1219-1228; and Cai et al. 2007 Sci. China Ser C-Life Sci 50:3:356-366 describe tetraploid rice having a PMeS genetic background.

### SUMMARY OF THE INVENTION

According to an aspect of some embodiments of the present invention there is provided a cultivated Oryza sativa plant having a partially or fully multiplied genome being at least as fertile as a diploid Oryza sativa plant isogenic to the genomically multiplied Oryza sativa plant when grown under the same conditions.

According to some embodiments of the invention, the plant is further characterized by at least one of:

(i) higher seed weight than that of a diploid Oryza sativa plant isogenic to the genomically multiplied Oryza sativa plant when grown under the same conditions;

(ii) higher crop yield, when compared to that of a diploid Oryza sativa plant isogenic to the genomically multiplied Oryza sativa plant when grown under the same conditions;

(iii) increased flag leaf width when compared to that of a diploid Oryza sativa plant isogenic to the genomically multiplied Oryza sativa plant when grown under the same conditions;

(iv) higher tiller number when compared to that of a diploid Oryza sativa plant isogenic to the genomically multiplied Oryza sativa plant when grown under the same conditions;

(v) higher photosynthetic efficiency when compared to that of a diploid Oryza sativa plant isogenic to the genomically multiplied Oryza sativa plant when grown under the same conditions.

According to some embodiments of the invention, the plant is further characterized by at least one of:

(i) at least 1.75 fold higher seed weight than that of a diploid Oryza sativa plant isogenic to the genomically multiplied Oryza sativa plant when grown under the same conditions;

(ii) at least 15% higher crop yield, when compared to that of a diploid Oryza sativa plant isogenic to the genomically multiplied Oryza sativa plant when grown under the same conditions;

(iii) at least 15% increased flag leaf width when compared to that of a diploid Oryza sativa plant isogenic to the genomically multiplied Oryza sativa plant when grown under the same conditions;

(iv) at least 15% higher tiller number when compared to that of a diploid Oryza sativa plant isogenic to the genomically multiplied Oryza sativa plant when grown under the same conditions;

According to some embodiments of the invention, the Oryza sativa plant and the plant isogenic thereto do not have a PMeS genetic background.

According to some embodiments of the invention, the oryza sativa is of a subspecies selected from the group consisting of Indica, Japonica, Aromatic and Glutinous.

According to an aspect of some embodiments of the present invention there is provided a hybrid plant having as a parental ancestor the plant described herein.

According to an aspect of some embodiments of the present invention there is provided a planted field comprising the plant described herein.

According to an aspect of some embodiments of the present invention there is provided a sown field comprising seeds of the plants described herein.

According to some embodiments of the invention, the plant is non-transgenic.

According to some embodiments of the invention, the fertility is exhibited at least on third generation of the cultivated Oryza sativa plant having the partially or fully multiplied genome.

According to some embodiments of the invention, the plant has a total grain number per plant ratio at least as similar to that of the diploid Oryza sativa plant under the same developmental stage and growth conditions.

According to some embodiments of the invention, the plant has a total plant length similar, higher or lower than that of the diploid Oryza sativa plant under the same developmental stage and growth conditions.

According to some embodiments of the invention, the fertility is determined by at least one of:

number of seeds per plant;

gamete fertility assay; and

acetocarmine pollen staining.

According to some embodiments of the invention, the plant is a tetraploid.

According to some embodiments of the invention, the plant is capable of cross-breeding with a diploid or tetraploid Oryza sativa.

According to an aspect of some embodiments of the present invention there is provided a plant part of the Oryza sativa plant described herein.

According to an aspect of some embodiments of the present invention there is provided a processed product of the plant or plant part described herein.

According to some embodiments of the invention, the processed product of is selected from the group consisting of food, feed, mushroom bed, mulching in horticultural crops, paper, compost, construction material and biofuel.

According to some embodiments of the invention, the food or feed is selected from the group consisting of puffed rice, flake rice, parched rice, noodles, cereals, cakes, breads, snacks, cookies, starch and biscuits.

According to an aspect of some embodiments of the present invention there is provided a meal produced from the plant or plant part described herein.

According to some embodiments of the invention, the plant part is a seed.

According to an aspect of some embodiments of the present invention there is provided an isolated regenerable cell of the Oryza sativa plant described herein.

According to some embodiments of the invention, the cell exhibits genomic stability for at least 3 passages in culture.

According to some embodiments of the invention, the cell of is from a mertistem, a pollen, a leaf, a root, a root tip, an anther, a pistil, a flower, a seed or a stem.

According to an aspect of some embodiments of the present invention there is provided a tissue culture comprising the regenerable cells.

According to an aspect of some embodiments of the present invention there is provided a method of producing Oryza sativa plant seeds, comprising self-breeding or cross-breeding the plant described herein.

According to an aspect of some embodiments of the present invention there is provided a method of developing a hybrid plant using plant breeding techniques, the method comprising using the plant described herein as a source of breeding material for self-breeding and/or cross-breeding.

According to an aspect of some embodiments of the present invention there is provided a method of producing Oryza sativa plant meal, the method comprising:
(a) harvesting grains of the Oryza sativa plant or plant part described herein; and
(b) processing the grains so as to produce the Oryza sativa meal.

According to an aspect of some embodiments of the present invention there is provided a method of generating a Oryza sativa plant seed having a partially or fully multiplied genome, the method comprising contacting the Oryza sativa plant seed with a G2/M cell cycle inhibitor under a transiently applied magnetic field thereby generating the Oryza sativa plant seed having a partially or fully multiplied genome.

According to some embodiments of the invention, the G2/M cell cycle inhibitor comprises a microtubule polymerization inhibitor.

According to some embodiments of the invention, the microtubule polymerization inhibitor is selected from the group consisting of colchicine, nocodazole, oryzaline, trifluraline and vinblastine sulphate.

According to some embodiments of the invention, the method further comprises subjecting the seed to a priming step prior to the contacting with the G2/M cell cycle inhibitor.

According to some embodiments of the invention, the priming step comprises sonicating the seed.

According to an aspect of some embodiments of the present invention there is provided a sample of representative seeds of an Oryza sativa plant having a partially or fully multiplied genome being at least as fertile as a diploid Oryza sativa plant isogenic to the genomically multiplied Oryza sativa plant when grown under the same conditions, wherein the sample has been deposited under the Budapest Treaty at the NCIMB under NCIMB 42084 (Indica/KR301-EP-4).

According to an aspect of some embodiments of the present invention there is provided a sample of representative seeds of an Oryza sativa plant having a partially or fully multiplied genome being at least as fertile as a diploid Oryza sativa plant isogenic to the genomically multiplied Oryza sativa plant when grown under the same conditions, wherein the sample of the cultivated Oryza sativa plant having the partially or fully multiplied genome has been deposited under the Budapest Treaty at the NCIMB under NCIMB 42084 (Indica/KR301-EP-4).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G are graphs showing the DNA content of the indicated genomically multiplied lines (FIGS. 1B-G) versus control diploid line 85 (FIG. 1A) as detected by Propidium Iodide staining and FACS analysis;

FIGS. 2A-E is a collection of photographs showing the size of the tetraploid seed (82-1) with respect to its isogenic diploid line (line 85);

FIGS. 3A-B show the PM-48M Photosynthesis Monitor (FIG. 3A) and the self-clamping leaf chambers LC-4A (FIG. 3B)

FIGS. 4A-B are photographs of three diploid leaves (FIG. 4A) and one leaf (FIG. 4B) of tetraploid rice in a photosynthesis efficiency assay. The leaf chambers (LC) are given as a reference for the size of the leaves.

FIG. 5 is a photograph showing the LC within the wind protection box. Note that the two LCs are placed back to back. In this manner when one LC is shaded the other one of the same treatment is lighted up.

Accordingly, at different positions during the same time of the day different PN values were obtained, within the canopy alongside irradiance and daylight spectra.

FIG. 6 is a graph showing the effect of genotype multiplication on the diurnal course of photosynthesis (conversion; 10 μMol CO₂ m⁻²s⁻¹=10 Kg CH₂O ha⁻¹ hr⁻¹). Full-line represents a polyploid rice, while the dashed line represents the isogenic diploid parent.

FIG. 7 is a graph showing cumulative PN as a function of the time of the day. Full-line represents a polyploid rice, while the dashed line represents the isogenic parent.

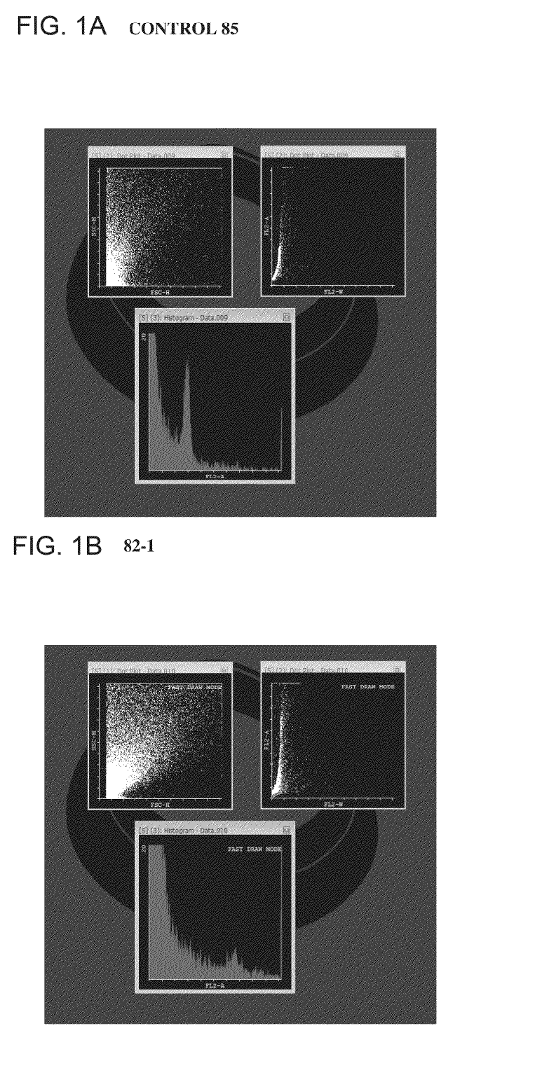

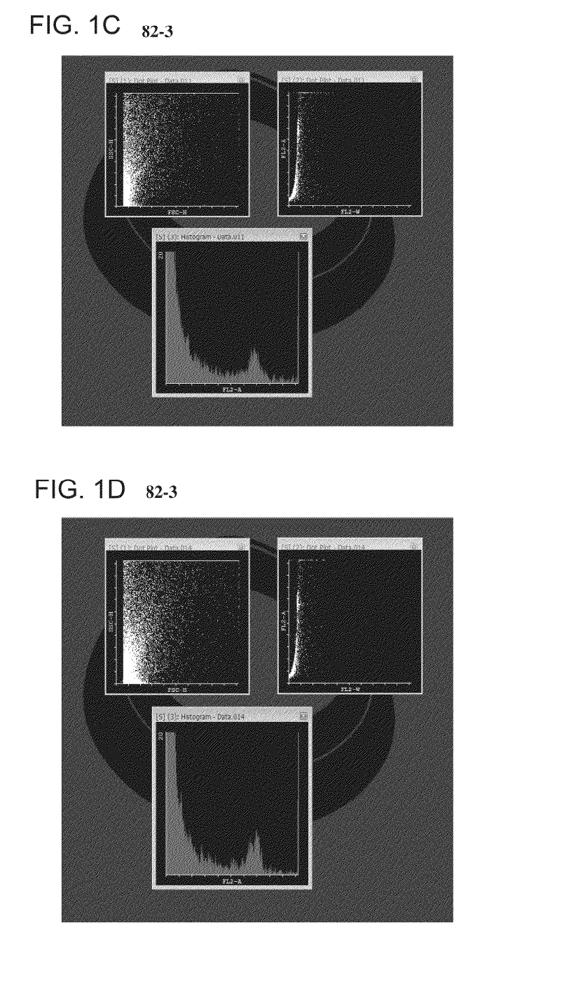

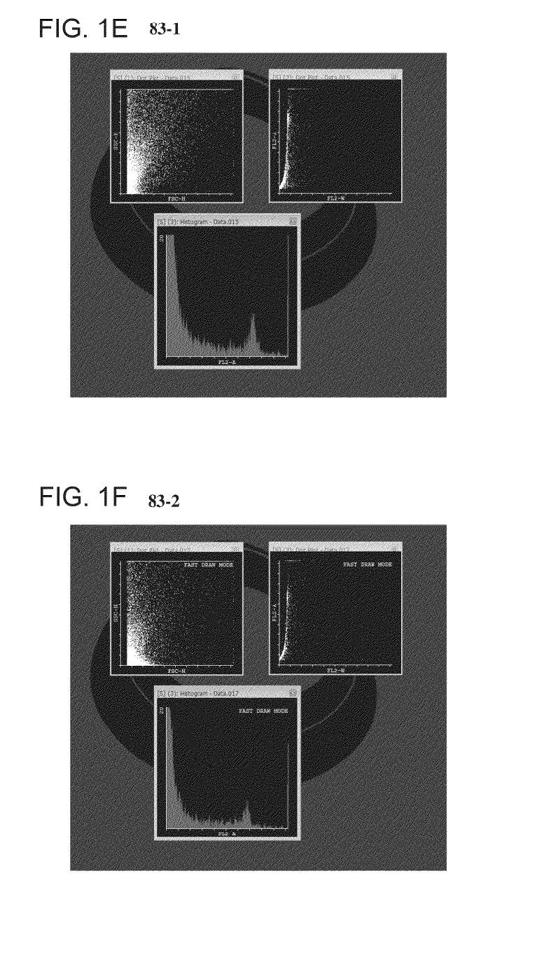

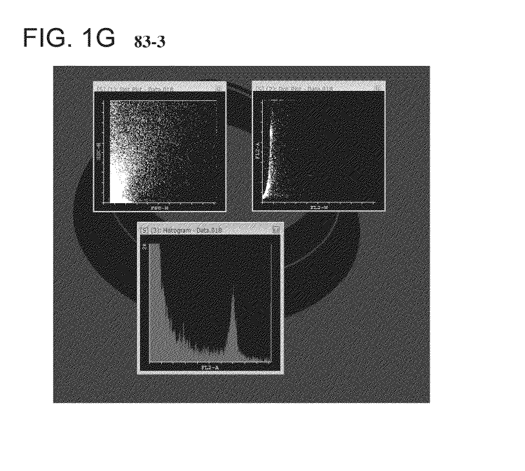

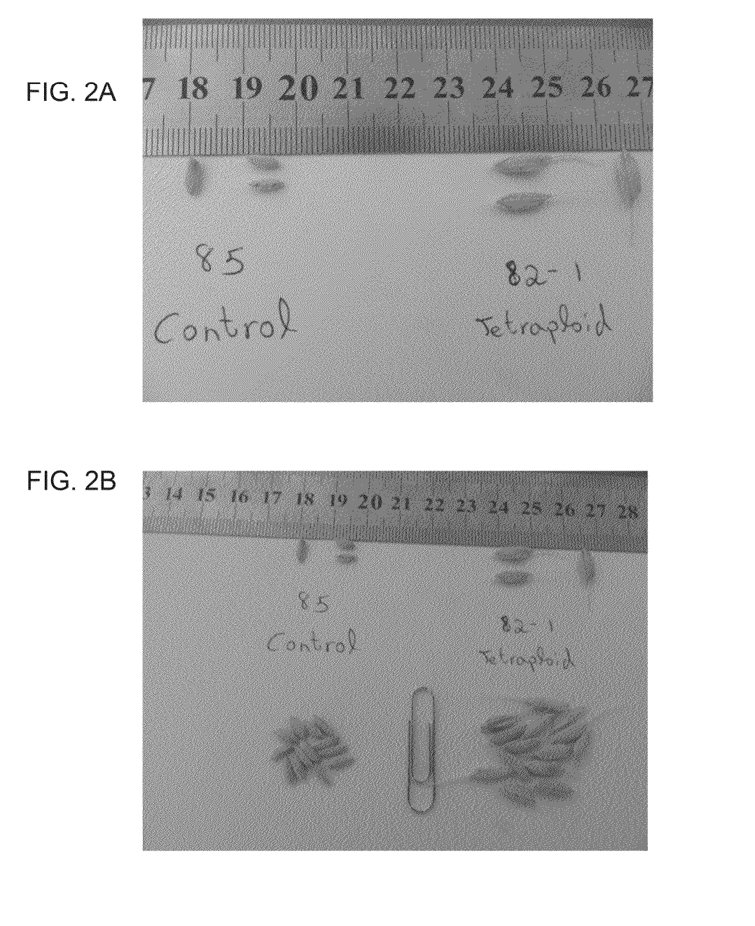

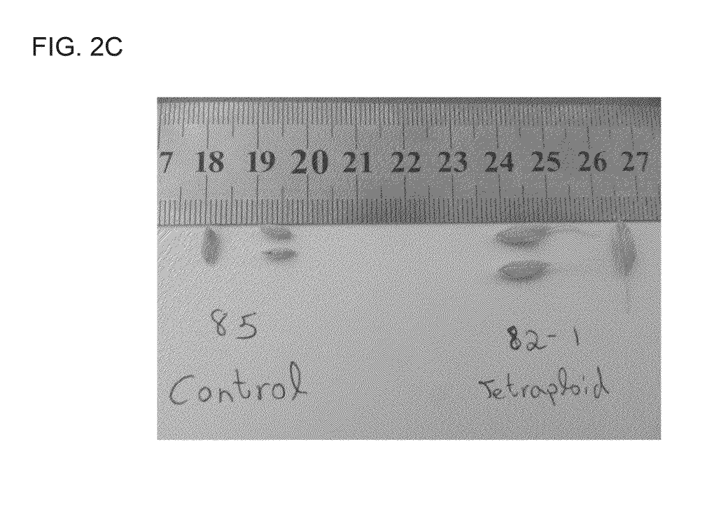

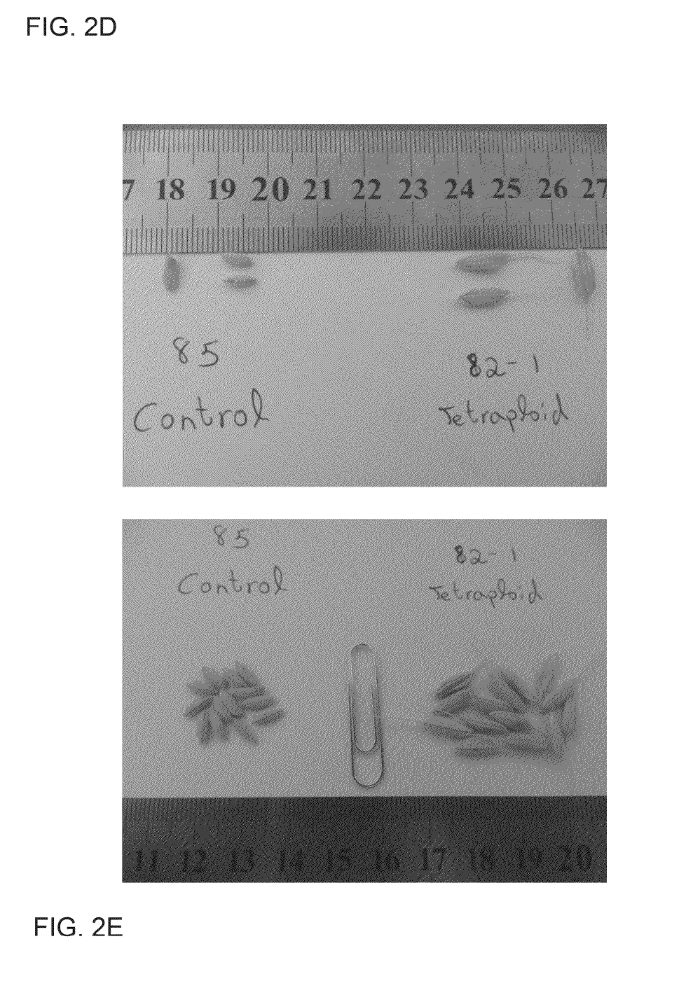

### DESCRIPTION OF SPECIFIC EMBODIMENTS OF THE INVENTION

The present invention, in some embodiments thereof, relates to a cultivated Oryza sativa plant having a partially or fully multiplied genome and uses of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Oryza sativa, is the plant species most commonly referred to in English as rice. Oryza sativa is the cereal with the smallest diploid genome, consisting of just 430 Mb across 12 chromosomes. It is renowned for being easy to genetically modify, and is a model organism for cereal biology. As a cereal grain, it is the most important staple food for a large part of the world's human population, especially in Asia and the West Indies. It is the grain with the third-highest worldwide production, after maize (corn) and wheat, according to data for 2009. It is a good source of vitamin B, fibre, and protein, but its main constituent is starch. This starch provides energy, which makes it a staple food.

A continuing goal of plant breeders is to develop stable high yielding Oryza sativa hybrids that are agronomically advantageous. The reasons for this are to maximize the amount of grain produced on the land used and to supply food for both animals and humans.

Induced polyploidy has been suggested for increasing Oryza sativa yields decades ago. To date, however, induced polyploidy has been successfully achieved for only a few Oryza sativa species that exhibited inferior fertility.

The present inventors have now designed a novel procedure for induced genome multiplication in Oryza sativa, resulting in plants that are genomically stable and fertile, at least as the isogenic diploid plant (progenitor plant). The induced polyploid plants are devoid of undesired genomic mutations and are characterized by seeds which are bigger in size and weigh more than those of the isogenic diploid plants. In addition that polyploid rice is characterized by increased flag leaf width, higher tiller number and higher photosynthesis efficiency than that of the isogenic diploid plant. In addition the polyploid plants showed an increase in yield per plant, vigor, fertility and biomass. For these reasons the Oryza sativa plants of some embodiments of the invention are considered of higher vigor and yield than that of the isogenic progenitor plant having a diploid genome (see Tables 2-5, below). These new traits may contribute to better climate adaptability and higher tolerance to biotic and abiotic stress. Furthermore, hybrid Oryza sativa seeds having the induced polyploid plants of the present invention as an ancestor parent may increase global Oryza sativa yields due to heterosis expression. In addition, the induced polyploid plant of some embodiments of the invention exhibits a similar or better fertility compared to that of the isogenic tetraploid progenitor plant already from early generations (e.g., first, second, third or fourth) following genome multiplication, negating the need for further breeding in order to improve fertility.

Thus, according to an aspect of the invention there is provided a cultivated Oryza sativa plant having a partially or fully multiplied genome being at least as fertile as a diploid Oryza sativa plant isogenic to the genomically multiplied Oryza sativa plant when grown under the same conditions.

As used herein, the term “Oryza sativa” or “rice” refers to the cultivated species Oryza sativa of the subfamily Bambusoideae or Ehrhartoideae of the family Poaceae or Gramineae of the tribe Oryzeae. The 2 cultivated species are Oryza sativa and Oryza glaberrima.

Rice varieties contemplated herein according to exemplary embodiments, refer to long grain, short grain, white, brown, red and black.

There are three main varieties of Oryza sativa:

Indica: The indica variety is long-grained, for example Basmati rice, grown notably on the Indian sub-continent.

Japonica: Japonica rice is short-grained and high in amylopectin (thus becoming “sticky” when cooked), and is grown mainly in more temperate or colder regions such as Japan.

Javonica: Javonica rice is broad-grained and grown in tropical climates.

Other major varieties include Aromatic and Glutinos.

According to a specific embodiment, the rice variety contemplated herein is Indica.

According to a specific embodiment, the rice variety contemplated herein is Japonica.

According to a specific embodiment, the rice variety contemplated herein is Indica basmatic.

Within each variety, there are many cultivars, each favored for particular purposes or regions. A japonica variety was the first to undergo genome sequencing, and is the focus of this landscape.

As used herein, the term “isogenic” refers to two individual plants (or portions thereof e.g., seeds, cells) having a substantially identical genotype (e.g., not more than 1 gene is different between the individuals).

According to a specific embodiment, the Oryza sativa sp. of the invention is cultivated Oryza sativa. As used herein, the term “cultivated” refers to domesticated Oryza sativa species, that were artificially selected by human. Oryza sativa species (the genetic source for genomic multiplication) of some embodiments of the invention are diploid in nature (n=12, 2n=24) and are primarily self-pollinated.

The following exemplary Oryza sativa species can be used in accordance with the present invention.
Oryza australiensis
Oryza barthii
Oryza glaberrima—African rice
Oryza latifolia
Oryza longistaminata
Oryza meridionalis
Oryza officinalis
Oryza punctata
Oryza rufipogon—brownbeard or red rice
Oryza sativa—Asian rice
Oryza nivara

Examples of some non-limiting commercial varieties are listed hereinbelow.

‘California Mochi Rice’

This type of rice is also known as sweet rice, glutinous rice, or waxy rice. These names are deceptive. Mochi rice is slightly sweeter than conventional rice, but the rice is not sweet and most palates would not detect any sweetness. The nature of the starch is almost pure amylopectin and so the rice is very sticky. (Every year near Christmas season several deaths are reported in Japan from people suffocating with mochi rice stuck in their throat.) Gluten is a type of protein that is very sticky (hence the name glutinous), but there is no gluten is rice. Mochi rice has many of the functional properties of waxy corn, which is also very high in amylopectin. Mochi rice is also a Japonica type of rice and has a gelatinization temperature of about 60 degrees centigrade and protein content of about 6.5%. Mochi rice is a specialty variety and a small number of acres in California are dedicated to this variety. Sage V Foods contracts directly with farmers for this variety. Sage V Foods was one of the pioneers in developing markets for this variety of rice in the U.S. and continues to control one of the largest acreage bases.

‘Thai Jasmine Rice’

Jasmine Rice from Thailand is an aromatic rice with a strong aroma and taste that is unique. The rice looks much like southern long grain rice before and after cooking, but the amylose content is around 18% and so the texture is sticky, much like California medium grain rice. The rice is best consumed after new crop is harvested. The rice hardens in texture and loses aroma with time. There are many varieties being grown in the U.S. in imitation of this unique type of rice. These varieties have improved over the years, but so far no one has matched the unique texture, aroma, and texture of Thai Jasmine.

‘Indian Basmati Rice’

Indian Basmati rice is also an aromatic rice, but has a very different aroma and taste from Thai Jasmine. Some people describe its aroma as popcorn like. This rice is grown in the northern Punjab region of India and Pakistan, and commands the highest price of any variety of rice grown in the world (not counting artificially high prices for rice in Japan). This rice has a high amylose content and a firm almost dry texture when properly cooked. The raw kernel is long and slender like southern long grain, but slightly smaller. The kernels increase in length by more than three times when cooked to produce a very long slender cooked grain. The best Indian Basmati has been aged for at least one year to increase firmness of cooked texture and increase the elongation achieved in cooking. Once again, there are many “knock off” varieties grown in the U.S., but none match authentic Indian Basmati for favor, aroma, texture, and appearance.

‘Arborio Rice’

Arborio Rice is an Italian variety of rice that is commonly used in risotto dishes.

It is close to California medium grain in appearance and texture. It is a bigger kernel with a distinct chalky center. When properly cooked, arborio rice develops a unique texture with a starchy creamy surface and a firm bite in the center. There are varieties of arborio rice grown in California that are as good as Italian varieties. Sage V Foods markets a California variety of arborio.

‘Wild Rice’

Wild rice in not officially classified as rice, but is in fact a different type of grass that grows a long stalk and thrives in deep water. It was traditionally grown wild in the lakes of northern United States and southern Canada. It is still grown this way in Minnesota and other northern areas. Indians harvested the rice in canoes, and then parched (primitive parboiling) the grains. Much of the wild rice from Minnesota is still harvested and parched with methods similar to the past. Parching gives the wild rice a strong flavor. All wild rice is sold with the bran on the kernel (like brown rice) and this gives it its black appearance. In California today, wild rice is mechanically farmed and harvested and then parboiled using modern methods. The quality of California rice is more consistent.

Specialty Varieties in the United States. In the U.S. more and more specialty rice varieties are being grown for niche markets. There are several varieties of rice that have been developed to perform like Thai Jasmine and Indian Basmati. There are several varieties of rice that have unusual bran colors like Wehani, red rice, and black rice. In California today there are several Japanese short grain varieties being grown like Akita Komachi and Koshi Hikari.

“A plant” refers to a whole plant or portions thereof (e.g., seeds, stems, fruit, leaves, flowers, tissues etc.), processed or non-processed [e.g., seeds, meal), dry tissue, cake etc.], regenerable tissue culture or cells isolated therefrom. According to some embodiments, the term plant as used herein also refers to hybrids having one the induced polyploid plants as at least one of its ancestors, as will be further defined and explained hereinbelow.

As used herein “partially or fully multiplied genome” refers to an addition of at least one chromosome (2n=or >25), or at least a complete chromosomal set (n=12) that result in a triploid plant (3n=36) or a full multiplication of the genome that results in a tetraploid plant (4N) or more.

The genomically multiplied plant of the invention is also referred to herein as “induced polyploid” plant.

According to a specific embodiment, the induced polyploid plant is 3N.

According to a specific embodiment, the induced polyploid plant is 4N.

According to a specific embodiment, the induced polyploid plant is 5N.

According to a specific embodiment, the induced polyploid plant is 6N.

According to a specific embodiment, the induced polyploid plant is 7N.

According to a specific embodiment, the induced polyploid plant is 8N.

According to a specific embodiment, the induced polyploid plant is 9N.

According to a specific embodiment, the induced polyploid plant is 10N.

According to a specific embodiment, the induced polyploid plant is 11N.

According to a specific embodiment, the induced polyploid plant is 12N.

According to a specific embodiment, the induced polyploid plant is not a genomically multiplied haploid plant.

As mentioned, the induced polyploid is at least as fertile (e.g., 90% or more) as the diploid Oryza sativa progenitor plant isogenic to the genomically multiplied Oryza sativa when grown under the same (i.e., identical) conditions and being of the same (i.e., identical) developmental stage. According to a specific embodiment, such a fertility level is achieved already after 3 generations following genome multiplication, but may also be exhibited already in the process such as at the first or second generations following genomic multiplication.

Of note the polyploid cultivated rice of the present teachings is in contrast to the nine known wild species of rice that are tetraploid.

As used herein the term “fertile” refers to the ability to reproduce sexually. Fertility can be assayed using methods which are well known in the art. Alternatively, fertility is defined as the ability to set seeds. The following parameters may be assayed in order to determine fertility: the number of seeds (grains); gamete fertility may be determined by pollen germination such as on a sucrose substrate; and alternatively or additionally acetocarmine staining, whereby a fertile pollen is stained.

As used herein the term “stable” or “genomic stability” refers to the number of chromosomes or chromosome copies, which remains constant through several generations (e.g., 3, 5 or 10), while the plant exhibits no substantial decline in at least one of the following parameters: yield (e.g., total seed weight/per plant or total seed weight/area unit), fertility, biomass, vigor.

According to a specific embodiment, stability is defined as producing a true to type offspring, keeping the variety strong and consistent.

According to an embodiment of the invention, the genomically multiplied plant is isogenic to the source plant, namely the diploid cultivated rice plant. The genomically multiplied plant has substantially the same genomic composition as the diploid plant in quality but not in quantity.

According to a specific embodiment, the plant exhibits genomic stability for at least 2, 3, 5, 10 or more passages in culture or generations of a whole plant.

According to a specific embodiment, the partially or fully multiplied plant (polyploid) of the invention, is further characterized by at least one of:

(i) higher seed weight than that of a diploid Oryza sativa plant isogenic to the genomically multiplied Oryza sativa plant when grown under the same conditions;

(ii) higher crop yield (yield per plant), when compared to that of a diploid Oryza sativa plant isogenic to the genomically multiplied Oryza sativa plant when grown under the same conditions;

(iii) increased flag leaf width when compared to that of a diploid Oryza sativa plant isogenic to the genomically multiplied Oryza sativa plant when grown under the same conditions;

(iv) higher tiller number when compared to that of a diploid Oryza sativa plant isogenic to the genomically multiplied Oryza sativa plant when grown under the same conditions;

(v) higher photosynthetic efficiency when compared to that of a diploid Oryza sativa plant isogenic to the genomically multiplied Oryza sativa plant when grown under the same conditions.

According to a specific embodiment, the partially or fully multiplied plant (polyploid) of the invention, is further characterized by at least one of:

(i) at least 1.75 fold higher seed weight than that of a diploid Oryza sativa plant isogenic to the genomically multiplied Oryza sativa plant when grown under the same conditions;

(ii) at least 15% higher crop yield (yield per plant), when compared to that of a diploid Oryza sativa plant isogenic to the genomically multiplied Oryza sativa plant when grown under the same conditions;

(iii) at least 15% increased flag leaf width when compared to that of a diploid Oryza sativa plant isogenic to the genomically multiplied Oryza sativa plant when grown under the same conditions;

(iv) at least 15% higher tiller number when compared to that of a diploid Oryza sativa plant isogenic to the genomically multiplied Oryza sativa plant when grown under the same conditions;

According to a specific embodiment, the polyploid plant is characterized by at least 2 of, i+ii, i+iii, i+iv, i+v, ii+iii, ii+iv+, ii+v, iii+iv, iii+v and iv+v.

According to a specific embodiment, the polyploid plant is characterized by at least 2 of, i+ii+iii, i+iii+iv, i+iii+v, ii+iii+iv.

According to specific embodiments, the plant is characterized by i+ii+iii+iv+v.

According to some embodiments of the present invention, a mature genomically multiplied plant has at least about the same (+/−10%) number of seeds as it's isogenic diploid progenitor grown under the same conditions; alternatively or additionally the genomically multiplied plant has at least 90% fertile pollen that are stained by acetocarmine; and alternatively or additionally at least 90% of seeds germinate on sucrose. The tetraploid plants generated according to the present teachings have total yield/plant which is higher by at least 10%, 15%, 20%, 25%, 30% or 35% than that of the isogenic progenitor plant. According to a specific embodiment, yield is measured using the following formula:

Yield per plant=total grain number/plant×grain weight

Comparison assays done for characterizing traits (e.g., fertility, yield, biomass and vigor) of the genomically multiplied plants of the present invention are typically performed in comparison to it's isogenic progenitor (hereinafter, “the diploid progenitor Oryza sativa plant” e.g., the “line 85”) when both are being of the same developmental stage and both are grown under the same growth conditions.

According to a specific embodiment, the genomically multiplied plant is characterized by a tiller number at least as similar to that of the diploid Oryza sativa isogenic progenitor plant of the same developmental stage and grown under the same growth conditions. According to a specific embodiment the tiller number is higher by, 5%, 10%, 15% or even more 20%, 25%, 30%, 35%, 40% or 50% (see Table 5).

According to a specific embodiment, the genomically multiplied plant is characterized by total grain weight per plant at least as similar to that of the diploid Oryza sativa isogenic progenitor plant of the same developmental stage and grown under the same growth conditions. According to a specific embodiment the total grain weight per plant is higher by at least 1.75 folds. 1.4-2 or 1.5-1.75, 1.75-2, 1.75-2.5, 1.8-2, 1.8-2.5. 1.95-3, 1.95-2.5.

According to a specific embodiment, the genomically multiplied plant is characterized by a grain weight at least as similar to that of the diploid Oryza sativa isogenic progenitor plant of the same developmental stage and grown under the same growth conditions. According to a specific embodiment the total grain weight per plant is higher by at least 1.75 folds. 1.4-2 or 1.5-1.75, 1.75-2, 1.75-2.5, 1.8-2, 1.8-2.5. 1.95-3, 1.95-2.5 (see Table 2).

According to a specific embodiment, the genomically multiplied plant is characterized by a total grain number per plant at least as similar to that of the diploid Oryza sativa isogenic progenitor plant of the same developmental stage and grown under the same growth conditions. According to a specific embodiment the total grain number per plant is higher by 10%, 15%, 20%, 25%, 30%, 35%, 40%, 45%, 50%, 60% or even more 80% or 90%.

According to a specific embodiment, the genomically multiplied plant is characterized by a pollen fertility at least as similar to that of the diploid Oryza sativa isogenic progenitor plant of the same developmental stage and grown under the same growth conditions. According to a specific embodiment the pollen fertility is 80%, 90%, 95% or even 100% identical to that of the diploid isogenic progenitor.

Interestingly, the plants of the invention are characterized by an above ground plant length that is similar or even higher than that of the isogenic progenitor plant of the same developmental stage and grown under the same growth conditions. According to a specific embodiment the plant length is higher by 2%, 3%, 4%, 5%, 6%, 7%, 8%, 9%, 10% or even 20%.

According to a specific embodiment, the genomically multiplied plant is characterized by increased flag leaf width than that of the diploid Oryza sativa isogenic progenitor plant of the same developmental stage and grown under the same growth conditions. According to a specific embodiment the flag leaf width is at least 15%, 17%, 20%, 22%, 25%, 30%, 40% or more increased as compared to the isogenic diploid progenitor (see Table 4).

Plants of the invention are characterized by at least one, two, three, four or all of higher biomass, yield, grain yield, grain yield per growth area, grain protein content, grain weight, stover yield, seed set, chromosome number, genomic composition, percent oil, vigor, insect resistance, pesticide resistance, drought tolerance, and abiotic stress tolerance than the diploid cultivated Oryza sativa plant isogenic thereto.

It will be appreciated that while a certain trait of the induced polyploid plant may be inferior with respect to the isogenic progenitor others can be superior thus providing an overall superior phenotype.

For example, the induced polyploid line or hybrid, may have a seed weight which is inferior with respect to that of the isogenic progenitor but seed weight/plant or growth area which is superior to that of the isogenic progenitor.

Likewise, the induced polyploid line or hybrid, may have a seed weight which is inferior with respect to that of the isogenic progenitor but protein content which is superior to that of the isogenic progenitor.

According to a specific embodiment, the polyploid plant of the invention or its isogenic progenitor is devoid of a PMeS genetic background as described in He et al. Planta 2010 232:1219-1228; and Cai et al. 2007 Sci. China Ser C-Life Sci 50:3:356-366 describe tetraploid rice having a PMeS genetic background. The PMeS genetic background is associated in alterations in a PH1-like gene as described in Griffiths 2006 Nature 439:749-752.

According to a specific embodiment, the plant is non-transgenic.

Oryza sative plant or part thereof (e.g., seed) prior to multiplication is typically diploid and not a haploid or double-haploid.

According to another embodiment, the plant is transgenic for instance by expressing a heterologous gene conferring agronomically beneficial traits such as pest resistance or morphological traits for cultivation. For example, the parent plant or the induced polyploid plant can express a transgene that is associated with improved nutritional value or disease tolerance. Rice transformation protocols are well known in the art. For instance see, Hiei et al. Plant Mol Biol. 1997 September; 35(1-2):205-1.

Genomically multiplied plant seeds of the present invention can be generated using an improved method of colchicination, as described below.

Thus, according to an aspect of the invention, there is provided a method of generating a Oryza sativa plant or part thereof (e.g., seed) having a partially or fully multiplied genome, the method comprising contacting the Oryza sativa plant or part thereof (e.g., seed) with a G2/M cell cycle inhibitor under a transiently applied magnetic field, thereby generating the Oryza sativa having a partially or fully multiplied genome.

Prior to treatment the seed is subjected to a priming step in the presence of NaCl:KNO₃. This step typically lasts for 12-48 hours (e.g., 24 h). The treatment is typically terminated when the length of the root reaches 1 cm.

A long soaking in water is preferably effected. Rice being grown in flooded fields can endure the long incubation in water (tap water). Thus according to a specific embodiment, the seeds are soaked in water for 10-25 hours e.g., 15-20 hours, e.g., 18 hours.

To improve permeability of the seeds to the treatment solution, the seeds are subjected to ultrasound treatment (e.g., 40-50 KHz e.g., 40 KHz, for 5 to 10 min e.g., 5 min) prior to contacting with the G2/M cycle inhibitor.

Wet seeds may respond better to treatment and therefore seeds are soaked in an aqueous solution (e.g., distilled water) at the initiation of treatment.

According to a specific embodiment, the entire treatment is performed in the dark and at room temperature (22-25° C.) or lower [e.g., for the ultrasound (US) stage].

Thus according to a specific embodiment, the seeds are soaked in water at room temperature and then subjected to US treatment in distilled water.

Once permeated, the seeds are placed in a receptacle containing the treatment solution and a magnetic field is turned on.

Typically, the G2/M cycle inhibitor comprises a microtubule polymerization inhibitor.

Examples of microtubule cycle inhibitors include, but are not limited colchicine, colcemid, trifluralin, oryzalin, benzimidazole carbamates (e.g. nocodazole, oncodazole, mebendazole, R 17934, MBC), o-isopropyl N-phenyl carbamate, chloroisopropyl N-phenyl carbamate, amiprophos-methyl, taxol, vinblastine, griseofulvin, caffeine, bis-ANS, maytansine, vinbalstine, vinblastine sulphate and podophyllotoxin.

The G2/M inhibitor is comprised in a treatment solution which may include additional active ingredients such as antioxidants. Specific examples of treatment solutions that can be used in accordance with the present teachings are listed below.

Of note the following ranges of microtubule polymerization inhibitors and antioxidants can be used in the treatment solution:

Of note the following ranges of microtubule polymerization inhibitors and antioxidants can be used in the treatment solution:

Microtubule polymerization inhibitor—0.1% Vinblastine sulphate, 0.1-0.5 mg/ml Colchicine, 0.002-0.005% Oryzalin.

Antioxidants—25 μg/ml Cyanidin 3-O-b-glucopyranoside, 10⁻⁶⁻10⁻⁴ M Baicalein, 10⁻⁶⁻10⁻⁴M Quercetin or 5 mM Trolox.

DNA protectants such as histones may be added to the solution.

The treatment solution may further comprise DMSO and detergents.

As mentioned, while treating the rice with a treatment solution which comprises the G2/M cycle inhibitor, the plant is further subjected to a magnetic field of at least 1000 gauss (e.g., 1550 Gauss) for about 2 hr. The seeds are placed in a magnetic field chamber such as that described in Example 1. After the indicated time, the seeds are removed from the magnetic field. During the treatment, the temperature does not exceed 24° C.

Once the seeds are removed from the magnetic field they are subject to a second round of treatment with the G2/M cycle inhibitor. Finally, the seeds are washed intensively to improve the germination rate and seeded on appropriate growth beds. Optionally, the seedlings are grown in the presence of Acadain™ (Acadian AgriTech) and Giberllon (the latter is used when treated with vinblastine, as the G2/M cycle inhibitor).

Using the above teachings, the present inventors have established genomically multiplied Oryza sativa plants.

Once established, the plants of the present invention can be propagated sexually or asexually such as by using tissue culturing techniques.

As used herein the phrase “tissue culture” refers to plant cells or plant parts from Oryza sativa grass can be generated, including plant protoplasts, plant calli, plant clumps, and plant cells that are intact in plants, or part of plants, such as seeds, leaves, stems, pollens, roots, root tips, anthers, ovules, petals, flowers, embryos, fibers and bolls.

According to some embodiments of the present invention, the cultured cells exhibit genomic stability for at least 2, 3, 4, 5, 7, 9 or 10 passages in culture.

Techniques of generating plant tissue culture and regenerating plants from tissue culture are well known in the art. For example, such techniques are set forth by Vasil., 1984. Cell Culture and Somatic Cell Genetics of Plants, Vol I, II, III, Laboratory Procedures and Their Applications, Academic Press, New York; Green et al., 1987. Plant Tissue and Cell Culture, Academic Press, New York; Weissbach and Weissbach. 1989. Methods for Plant Molecular Biology, Academic Press; Gelvin et al., 1990, Plant Molecular Biology Manual, Kluwer Academic Publishers; Evans et al., 1983, Handbook of Plant Cell Culture, MacMillian Publishing Company, New York; and Klee et al., 1987. Ann. Rev. of Plant Phys. 38:467 486.

The tissue culture can be generated from cells or protoplasts of a tissue selected from the group consisting of seeds, leaves, stems, pollens, roots, root tips, anthers, ovules, petals, flowers and embryos.

It will be appreciated that the plants of the present invention can also be used in plant breeding along with other Oryza sativa plants (i.e., self-breeding or cross breeding) such as with cultivated or wild Oryza sativa in order to generate novel plants or plant lines which exhibit at least some of the characteristics of the Oryza sativa plants of the present invention.

Plants resultant from crossing any of these with another plant can be utilized in pedigree breeding, transformation and/or backcrossing to generate additional cultivars which exhibit the characteristics of the genomically multiplied plants of the present invention and any other desired traits. Screening techniques employing molecular or biochemical procedures well known in the art can be used to ensure that the important commercial characteristics sought after are preserved in each breeding generation.

The goal of backcrossing is to alter or substitute a single trait or characteristic in a recurrent parental line. To accomplish this, a single gene of the recurrent parental line is substituted or supplemented with the desired gene from the nonrecurrent line, while retaining essentially all of the rest of the desired genes, and therefore the desired physiological and morphological constitution of the original line. The choice of the particular nonrecurrent parent will depend on the purpose of the backcross. One of the major purposes is to add some commercially desirable, agronomically important trait to the plant. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred. Likewise, transgenes can be introduced into the plant using any of a variety of established transformation methods well-known to persons skilled in the art, such as described above.

It will be appreciated that plants or hybrid plants of the present invention can be genetically modified such as in order to introduce traits of interest e.g. enhanced resistance to stress (e.g., biotic or abiotic).

Thus, the present invention provides novel genomically multiplied plants, hybrids, hybrids having as a parental ancestor a genomically multiplied Oryza sativa according to the above-teachings and cultivars, and seeds and tissue culture for generating same. Using the present teachings, the present inventors were able to generate a number of plant varieties which are induced polyploids. A sample of representative seeds has been deposited under the Budapest Treaty at the NCIMB Ltd. under NCIMB 42084 on Nov. 26, 2012. The NCIMB 42084 corresponds to the induced polyploid Indica/KR301-EP-4.

The plant of the present invention is capable of self-breeding or cross-breeding with a diploid or tetraploid Oryza sativa.

Thus, the present invention further provides for a hybrid plant having as a parental ancestor the genomically multiplied plant as described herein.

For instance, the male parent may be the genomically multiplied plant while the female parent may be a diploid Oryza sativa (4N×2N). Alternatively, two induced genomically multiplied plants of the same (e.g., 4N×4N, 6N×6N) or different ploidy (e.g., 6N×4N) can be crossed.

According to a specific embodiment the invention provides for a hybrid Oryza sativa (plant having a partially or fully multiplied genome.

The present invention further provides for a seed bag which comprises at least 10%, 20% 50% or 100% of the seeds of the plants or hybrid plants of the invention.

The present invention further provides for a planted or sown field which comprises any of the plants (or seeds) or hybrid plants (or seeds) of the invention.

Grains of the present invention are processed as meal used as supplements in foods or feed (e.g,. poultry and livestock).

Accordingly, the present invention further provides for a method of producing Oryza sativa meal, the method comprising harvesting grains of the plant or hybrid plant of the invention; and processing the grains so as to produce meal.

Specific uses of the plants, parts thereof and processed products contemplated according to some embodiments of the invention are further described hereinbelow.

Rice is a staple food and used by many ways as under:

Staple food: Rice is used as a staple food by more than 60 percent of world population. Cooking of rice is a most popular way of eating.

Starch: Rice starch is used in making ice cream, custard powder, puddings, gel, distillation of potable alcohol, etc.

Rice bran: It is used in confectionery products like bread, snacks, cookies and biscuits. The defatted bran is also used as cattle feed, organic fertilizer (compost), and medicinal purpose and in wax making.

Rice bran oil: Rice bran oil is used as edible oil, in soap and fatty acids manufacturing. It is also used in cosmetics, synthetic fibers, detergents and emulsifiers. It is nutritionally superior and provides better protection to heart.

Flaked rice: It is made from parboiled rice and used in many preparations.

Puffed rice: It is made from paddy and used as whole for eating.

Parched rice: It is made from parboiled rice and is easily digestible.

Rice husk: It is used as a fuel, in board and paper manufacturing, packing and building materials and as an insulator. It is also used for compost making and chemical derivatives.

Rice broken: It is used for making food item like breakfast cereals, baby foods, rice flour, noodles, rice cakes, etc. and also used as a poultry feed.

Rice straw: Mainly used as animal feed, fuel, mushroom bed, for mulching in horticultural crops and in preparation of paper and compost.

Paddy as a seed: The paddy is used as seed.

It is expected that during the life of a patent maturing from this application many relevant Oryza sativa varieties, Oryza sativa products and uses will be developed and the scope of the terms provided herein is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation and Qualification of Polyploid Rice Plants

Material and Methods

Genome Multiplication Procedure

Oryza sativa seeds, Indica Basmati, (referred to herein as the “85 control”) were treated prior to genome multiplication for 24 hours with an aerated solution of 1:1 NaCl:KNO₃, 8 ds/m. Seeds were then washed with tap water and allowed to air dry for 24 hours. For genome multiplication procedure, seeds were soaked in an aerated vessel full of water at 25° C. for 18 hours, transferred into a clean net bag and inserted into a distilled water-filled ultrasonic bath at a temperature of 25° C. Thereafter, sonication was performed (40 KHz) for 5 minutes at temperature below 26° C. The bag was placed into a vessel containing treatment solution at pH=6 which includes 0.5% DMSO, 5 drops/L TritonX100, microtubule polymerization inhibitor, antioxidant' 100 μg/ml Histones diluted in soften, nitrogen free water at 24° C.

Of note the following ranges of microtubule polymerization inhibitors and antioxidants can be used in the treatment solution:

Microtubule polymerization inhibitor—0.1% Vinblastine sulphate, 0.1-0.5 mg/ml Colchicine, 0.002-0.005% Oryzalin.

Antioxidants—25 μg/ml Cyanidin 3-O-b-glucopyranoside, 10⁻⁶⁻10⁻⁴ M Baicalein, 10⁻⁶⁻10⁻⁴M Quercetin or 5 mM Trolox.

Following a 2 hour incubation of the vessel within a magnetic field chamber (see Magnetic Field Chamber further below), seeds were removed from the bag and placed on top of a paper towels bed on a plastic tray, covered with a second layer of paper towel and soaked in the treatment solution for additional 24 hours at 25° C. (the paper towels bed kept wet for the whole incubation period). Seeds were collected and washed in a clean vessel with water (pH=7). All stages were performed in the dark.

Thereafter, treated seeds were placed on seedling tray containing soil supplemented with 35 ppm of 20:20:20 Micro Elements Fertilizer and moved to nursery using a day temperature range of 25-28° C., night range of 17-20° C. and minimal moisture of 40%. When using Vinblastine, 1% GIBERLLON was applied immediately after seeding. The following 3 weeks included treatment with ACADIAN™ twice a week.

Magnetic Field Chamber

The magnetic field chamber consisted of two magnet boards located 11 cm from each other. The magnetic field formed by the two magnets is a coil-shaped magnetic field with a minimal strength of 1550 gauss in its central solution (as described under genome multiplication procedure, above), and the bath was inserted into the magnetic chamber.

Ploidy Examination Using Flow Cytometry

Samples of nuclei for Flow cytometry were prepared from leaves. Each sample (1 cm²) was chopped with a razor blade in a chopping buffer consisting of, 9.15 g MgCl₂, 8.8 g sodium citrate, 4.19 g 3-[morpholino]propane sulfonic acid, 1 ml Triton X-100, 21.8 g sorbitol per liter. The resulting slurry was filtered through a 23 μm nylon mesh, and Propidium Iodide (PI) was added to a final concentration of 0.2 mg/L. The stained samples were stored on ice and analyzed by flow cytometry.

The flow cytometer was a FACSCalibur (BD Biosciences ltd.).

Breeder Selection

Genome multiplication protocol (as described above), was applied on “85 control” seeds. Plants were selected for high ploidy according to their phenotype in the field. The phenotypic analyses included a number of parameters including flag leaf width, tiller number, seed size. Thereafter, FACS analysis confirmed that the plants and their offspring were indeed of stable high ploidy.

TABLE 1
The Rice Polyploid Seeds as Evidenced by FACS Analysis.

| | | Ploidy | FACS Results in D2 | FACS Results in D3 | |
|---|---|---|---|---|---|
| Name | Generation | Level | Generation | Generation | Comments |
| 85 (Control) | F7+ | 2N | | | Rice |
| 82-1 | D3 | 4N | 680 | 640 | Stable High Ploidy Rice |
| 82-2 | D3 | 4N | 680 | 620 | Stable High Ploidy Rice |
| 83-2 | D3 | 4N | 680 | 620 | Stable High Ploidy Rice |
| 83-3 | D3 | 4N | 700 | 600 | Stable High Ploidy Rice |

Each plant family are the self-seeds of different successfully genome multiplied inflorence. Most of the families derived from different successfully genome multiplied Plants.

Self-crossing was employed to generate D2 and D3. D2 indicates that the plants are second generation after genome multiplication procedure. D3 indicates that the plants are third generation after the genome multiplication procedure.

The plants were self-crossed as follows; the inflorescence of the female plant was covered before the spikelets matured (before the stigmas were receptive). Of note, covering of the inflorescence ensures that the pollination is a self-pollination. The flowers are hermaphrodites (both male and female). Pollination occurs spontaneously. Seeds were harvested upon maturity.

Tables 2-5 illustrate phenotypic properties of the plants of some embodiments of the invention.

TABLE 2
Rice Polyploid Seeds Grain Weight Compared with Control Diploid
Isogenic Plants.

| | | | | Increased | Increased |
|---|---|---|---|---|---|
| | | | | weight compare | weight |
| | | | 1000 seeds | to control | compared to |
| Name | Generation | Ploidy Level | weight | (fold change) | control (%) |
| 85 (Control) | F7+ | 2n | 18.9 | | |
| 82-1 | D3 | 4n | 37.7 | 1.99 | 199 |
| 82-2 | D3 | 4n | 37.3 | 1.97 | 197 |
| 83-2 | D3 | 4n | 44.1 | 2.33 | 133 |
| 83-3 | D3 | 4n | 40.5 | 2.14 | 214 |

TABLE 3
Crop Yield of Rice Polyploid Plants Compared to Control Diploid
Isogenic Plants.

| | | | Increased | |
|---|---|---|---|---|
| | | | yield | Increased yield |
| | | | compare | compare to |
| | | Yield | to control | control |
| Name | Generation | (Ton/Ha) | (fold change) | (%) |
| 85 (Control) | F7+ | 9554 | | |
| 82-1 | D3 | 11,244 | 1.18 | 118 |
| 82-2 | D3 | 14,892 | 1.56 | 156 |
| 83-2 | D3 | 15,997 | 1.67 | 167 |
| 83-3 | D3 | 12,241 | 1.28 | 128 |

Crop yield of the polyploid plants increased by average fold of 1.4 compared to diploid control plants.

TABLE 4
Flag Leaf Width of Polyploid Rice Plant and Control Plant.

| | | | Increased flag | Increased flag |
|---|---|---|---|---|
| | | | leaf width | leaf width |
| | | Flag Leaf | compare | compare to |
| | | Width | to control | control |
| Name | Generation | (cm) | (fold change) | (%) |
| 85 (Control) | F7+ | 1.3 | | |
| 82-1 | D3 | 1.6 | 1.2 | 120 |
| 82-2 | D3 | 1.6 | 1.2 | 120 |
| 83-2 | D3 | 1.8 | 1.4 | 140 |
| 83-3 | D3 | 1.7 | 1.3 | 130 |

TABLE 5
Tiller Number of Polyploid Rice Plant and Control Plant.

| | | | Increased | Increased |
|---|---|---|---|---|
| | | | tiller No. | tiller No. |
| | | | compare | compare |
| | | Tiller Number | to control | to control |
| Name | Generation | (cm) | (fold change) | (%) |
| 85 (Control) | F7+ | 102 | | |
| 82-1 | D3 | 107 | 1.2 | 120 |
| 82-2 | D3 | 123 | 1.2 | 120 |
| 83-2 | D3 | 92 | 1.4 | 140 |
| 83-3 | D3 | 94 | 1.3 | 130 |

### Example 2

### The Effect of Multiplied Rice Genotype on Plant Productivity, Carbon Dioxide Sequestration and Quantum Use Efficiency

Materials and Methods

Study region: A typical Mediterranean climate prevails in the study region with the long term mean annual temperature 18-20° C. (Minimum and maximum air temperatures are -8-10° C. in January and 30-35° C. in August), 600-700 mm precipitation falls during the winter (November to May)., 1500-2000 mm potential evapotranspiration and incident PAR of 300-350 MJ m⁻² per month. Maximum incident PAR occurs in August (410-420 MJ m⁻² month⁻¹) and the minimum in December (140-160 MJ m⁻² month⁻¹).

The surface soils (0-30 cm) with different proportions of sand, silt, and clay fractions in the study locations were predominantly fine-textured soils displayed a narrow range of variation with respect to field capacity and permanent wilting point which corresponded to −0.03 MPa and −1.5 MPa, respectively, in matric potential. Soil dry bulk densities ranged between 1.22 and 1.35 g cm⁻³. The soils had no salinity problem, and total soluble salt contents were under 0.1%. Soils of the study sites had slightly alkaline pH values of 7.5 to 7.7 and were determined to be poor in soil organic matter (1.18 to 2.37%).

Measurements: The coupled dynamics of net photosynthetic rate (P_{N}), transpiration rate (E_{T}), water use efficiency (WUE), light use efficiency (LUE), stomatal conductance (gₛ), photosynthetically active radiation (PAR), air temperature (T), relative humidity (RH), and atmospheric CO₂ concentration (Cₐₜₘ) were measured.

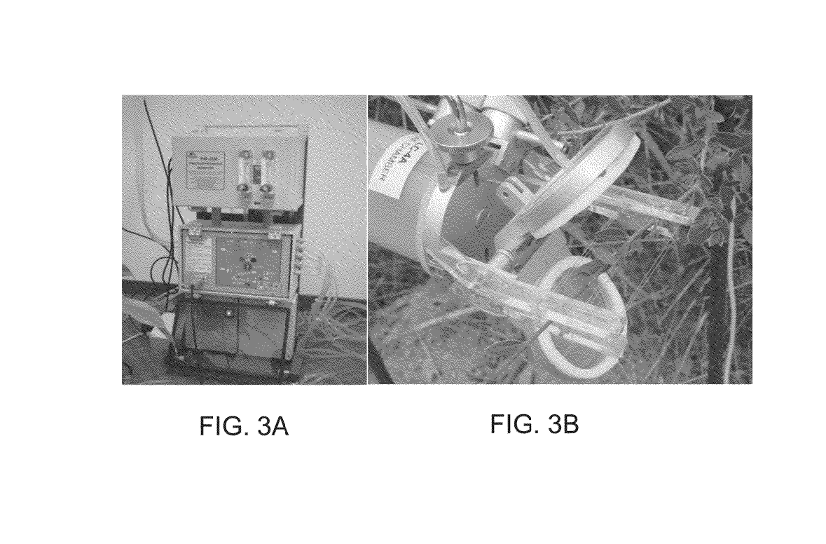

For measuring various environmental and plant parameters the PTM-48A Photosynthesis Monitor (Photosynthesis and Transpiration Monitor Bio Instruments SRL, Chisinau, Moldova) wwwdotphyto-sensordotcom) was used (FIG. 3A).

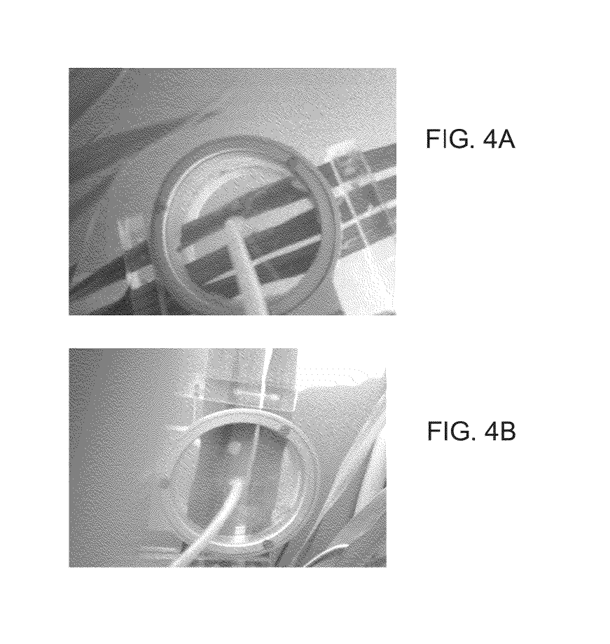

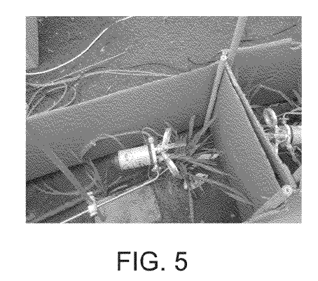

The system contains: four self-clamping leaf chambers LC-4A (FIG. 4B) which successively close for two minutes for monitoring CO₂ exchange of leaves, infrared CO₂ analyzer and a built-in data logger. The monitor also has eleven inputs for additional sensors.

The additional sensors used in the current experiment included:

PIR-1 Photosynthesis Radiation Sensor ATH-2 Air Temperature and Humidity Sensor

Photosynthesis and transpiration data from four leaf chambers as well as data from the additional sensors were automatically recorded every 30 minutes around-the-clock.

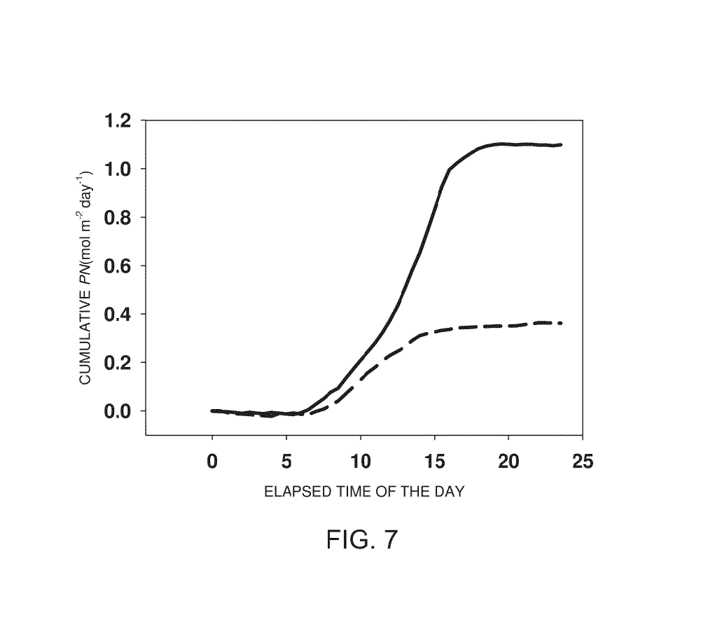

Statistical considerations: The average of two leaf chambers per diploid plant was used and two chambers used to measure the multiplied rice genotypes. Ambient CO₂ was detected with four probes and the standard deviation of the output was calculated. . The standard deviation of photosynthesis was calculated on from 6 PN data in each hour. The source of the data came from 2 LC;s per treatment times 3 consecutive measuring days that climatically did not differ from one another. The largest coefficient of variation (CV) was 0.35 for the two treatments and it was calculated during the peak PN values. It should be mentioned that measurements were made within a wind protection box (FIG. 7).

Results

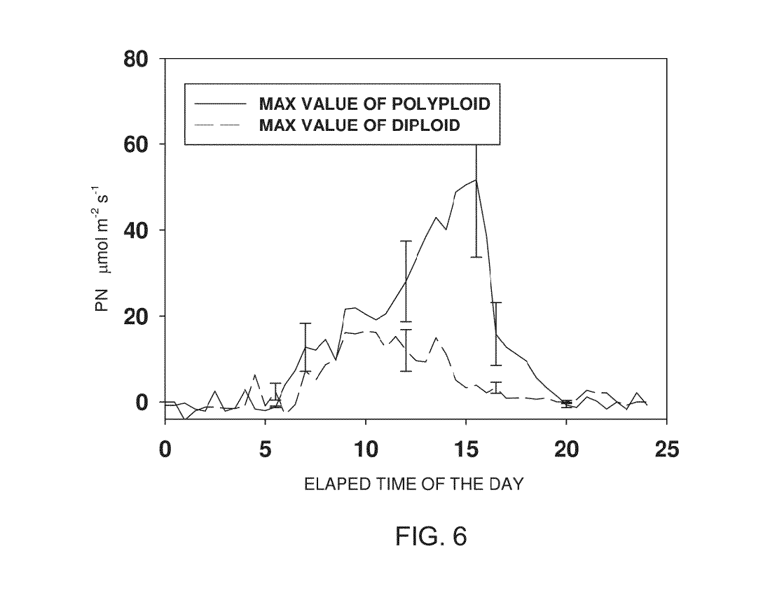

The results in FIG. 6 display the daily course of photosynthesis by diploid and tetraploid rice. From FIG. 6 maximum average photosynthetic rates were about 52 μmol m⁻² s⁻¹ for the tetraploids and only 15 μmol m⁻² s⁻¹ for the diploids. The peaks of photosynthesis were at about 3 pm.

The cumulative PN of diploid and tetraploid rice is displayed in FIG. 7.

From FIG. 7, it is evident that the total net production of tetraploid was about 32 while for the diploid it was only 11 g CH₂O m⁻² day⁻¹.

Acclimation of tetraploid rice to high temperatures and PPFD (or irradiance) is better than that of diploid. More over under relatively harsh conditions the tetraploid rice can maximize the rate of photosynthetic carbon assimilation the photosynthetic efficiency of the tetraploid remains high.

### Effect of Multiplied Genotypes on Biomass Production

Discussion

This study was aimed to study the effect of multiplied rice genotype on its productivity, its ability to sequestrate CO₂ and its quantum use efficiency. Its unique approach was that with the appropriate field instrumentation it provided increased understandings of the complex interactions between crops and their environment. Undoubtedly the models for predicting effect of high temperature are constantly modified as knowledge increases but this study with the understanding of the interactive effects was a step to improve and demonstrate the advantages of multiplyed genotypes and the economic benefit that can be obtained.

Results showed that values for continuous, instantaneous photosynthesis and cumulative CO₂ sequestration were 3-4 times higher in the tetraploid variety compare to the diploid variety.

The effect of the genotype: maximum average PN rates were about 52 and μmol m⁻² s⁻¹ for the tetraploids and only 16 μmol m⁻² s⁻¹ for the diploids. The peaks of photosynthesis were at 3:30 pm for the tetraploid and at 9 am for the diploid. The total net production of tetraploid was about 32 while for the diploid it was only 11 g CH₂O m⁻² day⁻¹.

The field was at high water content and well irrigated to enabled full stomatal activity and as a result the measured PN especially by the tetraploid Rice was very high

The effect of the environmental conditions: Maximum and minimum temperature during the measuring period was 36° C. and 17° C. respectively Photosynthetic process of the diploid Rice peaked at about 27° C. and at Photosynthetic Photon Flux density (PPFD) of 925 μmol m⁻² s⁻¹ On the other hand the tetraploid Rice peaked at 33° C. and PPFD of 1677 μmol m⁻² s⁻¹ and stopped at about 38° C. Maximum PN of the tetraploid tomatoe (52 μMol m⁻² s⁻¹) was 3 times higher than that of diploid tomatoe (15 μMol m⁻² s^{-1).} Note that reported maximum light-saturated rates of photosynthesis (PNₘₐₓ) expressed per unit leaf surface as a C3 plant was about 70 μMol m⁻² s⁻¹ (Murchie, et al. 2002). It means that tetraploidity is enhancing photosynthetic quantum use efficiency from 0.01 μMol m⁻² s⁻¹ per 1 PPFD unit to 0.03 μMol m⁻² s⁻¹ per 1 PPFD unit and that it is more resistant to high temperatur than the tetraploid. This experiment indicated that increases in ambient temperature can then lead to significant decrease in net CO₂ uptake ability of the diploid . Moreover, in arid climates the tetraploid rice as a representative for other multiplied genotype plant can improve carbon sequestration while increasing the yield.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A cultivated Oryza sativa plant having a partially or fully multiplied genome being at least as fertile as a diploid Oryza sativa plant isogenic to said genomically multiplied Oryza sativa plant when grown under the same conditions.

2. The plant of claim 1, further characterized by at least one of:
(i) higher seed weight than that of a diploid Oryza sativa plant isogenic to said genomically multiplied Oryza sativa plant when grown under the same conditions;
(ii) higher crop yield, when compared to that of a diploid Oryza sativa plant isogenic to said genomically multiplied Oryza sativa plant when grown under the same conditions;
(iii) increased flag leaf width when compared to that of a diploid Oryza sativa plant isogenic to said genomically multiplied Oryza sativa plant when grown under the same conditions;
(iv) higher tiller number when compared to that of a diploid Oryza sativa plant isogenic to said genomically multiplied Oryza sativa plant when grown under the same conditions;
(v) higher photosynthetic efficiency when compared to that of a diploid Oryza sativa plant isogenic to said genomically multiplied Oryza sativa plant when grown under the same conditions;
(vi) at least 1.75 fold higher seed weight than that of a diploid Oryza sativa plant isogenic to said genomically multiplied Oryza sativa plant when grown under the same conditions:
(vii) at least 15% higher crop yield. when compared to that of a diploid Oryza sativa plant isogenic to said genomicallv multiplied Oryza sativa plant when grown under the same conditions;
(viii) at least 15% increased flag leaf width when compared to that of a diploid Oryza sativa plant isogenic to said genomically multiplied Oryza sativa plant when grown under the same conditions;
(ix) at least 15% higher tiller number when compared to that of a diploid Oryza sativa plant isogenic to said genomically multiplied Orysa sativa plant when grown under the same conditions;
(x) having a total grain number per plant ratio at least as similar to that of said diploid Oryza sativa plant under the same developmental stage and growth conditions;
(xi) having a total plant length similar or lower than that of said diploid Oryza sativa plant under the same developmental stage and growth conditions;
(xii) capability of cross-breeding with a diploid or tetraploid Oryza sativa; and
(xiii) grain yield per growth area at least as similar to that of said diploid Oryza sativa plant isogenic to said genomically multiplied Oryza sativa plant when grown under the same devepmental stage and growth conditions.

3. (canceled)

4. The plant of claim 1, wherein said Oryza sativa plant and said plant isogenic thereto do not have a PMeS genetic background.

5. (canceled)

6. A hybrid plant having as a parental ancestor the plant of claim 1.

7-8. (canceled)

9. The plant of claim 1 being non-transgenic.

10. The plant of claim 1, wherein said fertility is exhibited at least on third generation of said cultivated Oryza sativa plant having said partially or fully multiplied genome.

11-12. (canceled)

13. The plant of claim 1, wherein said fertility is determined by at least one of:
number of seeds per plant;
gamete fertility assay; and
acetocarmine pollen staining.

14. The plant of claim 1, being a tetraploid.

15. (canceled)

16. A plant part of the Oryza sativa plant of claim 1 and optionally wherein said plant part is a seed.

17. A processed product of the plant or plant part of claim 1.

18-19. (canceled)

20. A meal produced from the plant of claim 1.

21. (canceled)

22. An isolated regenerable cell of the Oryza sativa plant of claim 1 and optionally wherein the cell exhibits genomic stability for at least 3 passages in culture.

23-25. (canceled)

26. A method of producing Oryza sativa plant seeds, comprising self-breeding or cross-breeding the plant of claim 1.

27. A method of developing a hybrid plant using plant breeding techniques, the method comprising using the plant of claim 1 as a source of breeding material for self-breeding and/or cross-breeding.

28. A method of producing Oryza sativa plant meal, the method comprising:
(a) harvesting grains of the Oryza sativa plant or plant part of claim 1; and
(b) processing said grains so as to produce the Oryza sativa meal.

29. A method of generating an Oryza sativa plant seed having a partially or fully multiplied genome, the method comprising contacting the Oryza sativa plant seed with a G2/M cell cycle inhibitor under a transiently applied magnetic field thereby generating the Oryza sativa plant seed having a partially or fully multiplied genome.

30. The method of claim 29, wherein said G2/M cell cycle inhibitor comprises a microtubule polymerization inhibitor and optionally wherein said microtubule polymerization inhibitor is selected from the group consisting of colchicine nocodazole, oryzaline, trifluraline and vinblastine sulphate.

31. (canceled)

32. The method of claim 29, further comprising subjecting the seed to a priming step prior to said contacting with said G2/M cell cycle inhibitor and optionally wherein said priming step comprises sonicatin said seed.

33-35. (canceled)

36. An Oryza sativa seed obtainable according to the method of claim 29.
